(19) United States
(12) Patent Application Publication
(10) Pub. No.: US 2011/0027760 A1
(43) Pub. Date: Feb. 3, 2011

(54) DENTAL FILLING COMPOSITION COMPRISING ACIDIC POLYMER COMPOUND AND METHOD OF USING THE SAME

(76) Inventors: Prabhakara S. Rao, Maplewood, MN (US); Steven M. Aasen, Woodbury, MN (US); Belma Erdogan-Haug, St. Paul, MN (US); Russell A. Roiko, Rogers, MN (US); Eugene G. Joseph, Blacksburg, VA (US)

Correspondence Address: 3M INNOVATIVE PROPERTIES COMPANY, PO BOX 33427, ST. PAUL, MN, 55133-3427, US

(21) Appl. No.: 12/937,302

(22) PCT Filed: Apr. 6, 2009
(86) PCT No.: PCT/US09/39599

§ 371 Date: Oct. 11, 2010

Related U.S. Application Data
(60) Provisional application No. 61/046,013, filed on Apr. 18, 2008.

(51) Int. Cl. A61C 5/04; C08L 33/10; C08L 43/04; A61K 6/083

(52) U.S. Cl. 433/228.1; 525/55; 525/209; 525/222; 523/116; 523/117

(57) ABSTRACT

Compositions comprising an acidic polymer compound and a (meth)acrylic copolymer prepared from reactants comprising at least one aliphatic, aromatic, or aralkyl (meth)acrylate monomer and at least one ethylenically unsaturated monomer having a polar group or a siloxane group, and dental filling compositions comprising the composition, and methods of using the compositions

This application claims the benefit of U.S. Provisional Application No. 61/046,013, filed Apr. 18, 2008, which is incorporated herein by reference in its entirety.

### BACKGROUND

The practice of endodontics includes the treatment of diseased root canals, typically when a tooth is intact but the root or pulp tissue is diseased. Access to the root canal has been made by drilling an opening in a tooth surface. Subsequently, the root material has been removed from the root canal, and the canal has been enlarged and then filled.

Root canal filling materials have been made of natural rubbers, for example gutta percha. In some instances, the gutta percha filling materials have been placed, in the form of cylinders or cones, into root canals. The filling materials have then been compressed or heated. More recently, the gutta percha filling materials have been softened by heating using a “gun” which has then been used to force the filling material into the root canal. Root canal filling materials comprising gutta percha have been used in combination with dental or endodontic sealing materials to seal the root canal around the filling material.

### SUMMARY

There is a need for compositions for filling dental cavities such as root canals, which have useful physical properties such as a low melting or softening temperature, sufficiently low viscosity when melted or softened to flow or be compacted into a root canal, and resistance to biological degradation.

In one aspect, a composition is provided comprising an acidic polymer compound and a (meth)acrylic copolymer. The (meth)acrylic copolymer is prepared from reactants comprising at least one aliphatic, aromatic, or aralkyl meth(acrylate) monomer and at least one ethylenically unsaturated monomer having a polar group or a siloxane group. The (meth)acrylic copolymer has a glass transition temperature no greater than 60° C.

In another aspect, a composition is provided comprising an acidic polymer compound comprising a polymer prepared from reactants comprising ethylene or an alpha-olefin, and a (meth)acrylic copolymer. The (meth)acrylic copolymer is prepared from reactants comprising at least one C8 to C18 alkyl meth(acrylate) monomer; and at least one ethylenically unsaturated monomer having a polar group or a siloxane group. The (meth)acrylic copolymer has a glass transition temperature no greater than 10° C.

In yet another aspect, a method of restoring a dental cavity is provided, the method comprising providing a composition comprising an acidic polymer compound and a (meth)acrylic copolymer. The (meth)acrylic copolymer is prepared from reactants comprising at least one aliphatic, aromatic, or aralkyl meth(acrylate) monomer and at least one ethylenically unsaturated monomer having a polar group or a siloxane group. The (meth)acrylic copolymer has a glass transition temperature no greater than 60° C. The method further comprises inserting the composition into the dental cavity.

In still another aspect, an article for filling a root canal is provided, comprising an acidic polymer compound and a (meth)acrylic copolymer. The (meth)acrylic copolymer is prepared from reactants comprising at least one aliphatic, aromatic, or aralkyl meth(acrylate) monomer and at least one ethylenically unsaturated monomer having a polar group or a siloxane group. The (meth)acrylic copolymer has a glass transition temperature no greater than 60° C. The article has an aspect ratio of at least 2 to 1.

### DETAILED DESCRIPTION

In several places throughout the application, guidance is provided through lists of examples, which examples can be used in various combinations. In each instance, the recited list serves only as a representative group and should not be interpreted as an exclusive list.

Any recitation of numerical ranges by endpoints includes all numbers subsumed within that range (e.g., 1 to 5 includes 1, 1.5, 2, 2.75, 3, 3.80, 4, 5, etc.).

The terms “a,” “an,” “the,” “at least one,” and “one or more” are used interchangeably. Thus, for example, a composition that comprises “a” compound of Formula I can be interpreted to mean that the composition includes “one or more” compounds of Formula I.

The term “acidic polymer compound” refers to an acidic polymer (e.g., an acidic olefin addition polymer) or to a mixture of a polymer (e.g., an olefin addition polymer) and an acidic compound (e.g., a fatty carboxylic acid).

The composition comprises an acid polymer compound that can comprise an acidic olefin addition polymer. The acidic olefin addition polymer can be prepared from reactants comprising any olefin. The olefin can comprise, for example, ethylene, propylene, or a combination of ethylene and propylene.

In some embodiments, the olefin comprises an alpha-olefin. The olefin can comprise any alpha-olefin. Non-limiting examples of alpha-olefins include 1-butene, 1-pentene, 1-hexene, 1-heptene, 1-octene, 1-nonene, 1-decene, 1-undecene, 1-dodecene, 1-tridecene, 1-tetradecene, 1-hexadecene, and 1-octadecene. The alpha-olefin can be a linear alpha-olefin (i.e., the alpha-olefin has a linear structure) or a branched alpha-olefin (i.e., the alpha-olefin comprises a branched structure). In some embodiments, the alpha-olefin is a linear alpha-olefin.

The acidic olefin addition polymer can be prepared from reactants comprising ethylene and an alpha-olefin (i.e., the acidic olefin addition polymer can be an acidic olefin addition copolymer). Such polymers can be referred to as “alpha olefin polymers.” Such an acidic olefin addition polymer can be prepared from reactants comprising any mole percentage of alpha-olefin. For example, the acidic olefin addition polymer can be prepared from reactants comprising at least 0.1 mole percent, 0.5 mole percent, at least 1 mole percent, at least 2 mole percent, at least 3 mole percent, at least 4 mole percent, at least 5 mole percent, at least 6 mole percent, at least 7 mole percent, at least 8 mole percent, at least 9 mole percent, at least 10 mole percent, at least 12 mole percent, or at least 15 mole percent alpha-olefin. The acidic olefin addition polymer can be prepared from reactants comprising no greater than 25 mole percent, no greater than 20 mole percent, no greater than 15 mole percent, no greater than 12 mole percent, no greater than 10 mole percent, no greater than 9 mole percent, no greater than 8 mole percent, no greater than 7 mole percent, no greater than 6 mole percent, no greater than 5 mole percent, no greater than 4 mole percent, no greater than 4 mole percent, no greater than 3 mole percent, no greater than 2 mole percent, or no greater than 1 mole percent alpha-olefin.

The acidic olefin addition polymer can be prepared from reactants comprising at least one acidic monomer or at least one acid-precursor monomer. An acidic monomer can comprise an acidic functional group, e.g., a carboxylic acid group. An acid-precursor monomer can comprise a functional group that can react (e.g., hydrolyze) to form an acid such as a carboxylic acid. Such a reaction can be carried out with a polymer prepared from reactants comprising at least one acid-precursor monomer. Non-limiting examples of acidic monomers include acrylic acid, methacrylic acid, maleic acid, and itaconic acid. Non-limiting examples of acid-precursor monomers include maleic anhydride and itaconic anhydride. In some embodiments, acidic olefin addition polymer can be prepared from reactants comprising at least one acidic monomer and at least one acid-precursor monomer.

An acidic olefin addition polymer prepared from reactants comprising at least one acidic monomer and/or at least one acid-precursor monomer can be prepared from reactants comprising any mole percentage of acidic monomer or acid-precursor monomer. An acidic olefin addition polymer can be prepared from reactants comprising at least 0.1 mole percent, at least 0.2 mole percent, at least 1 mole percent, at least 2 mole percent, at least 5 mole percent, at least 10 mole percent, at least 15 mole percent, at least 20 mole percent, at least 25 mole percent, at least 30 mole percent, at least 35 mole percent, at least 40 mole percent, at least 45 mole percent, at least 50 mole percent, at least 55 mole percent, or at least 60 mole percent acidic monomer and/or acid-precursor monomer. An acidic olefin addition polymer can be prepared from reactants comprising no greater than 1 mole percent, no greater than 2 mole percent, no greater than 5 mole percent, no greater than 10 mole percent, no greater than 15 mole percent, no greater than 20 mole percent, no greater than 25 mole percent, no greater than 30 mole percent, no greater than 35 mole percent, no greater than 40 mole percent, no greater than 45 mole percent, no greater than 50 mole percent, no greater than 55 mole percent, no greater than 60 mole percent, no greater than 65 mole percent, no greater than 70 mole percent, or no greater than 75 mole percent acidic monomer and/or acid-precursor monomer.

Non-limiting examples of acidic olefin addition polymers include poly(ethylene-co-methacrylic acid (comprising, for example, 1 weight percent, 2 weight percent, 4 weight percent, 9 weight percent, or 12 weight percent methacrylic acid), poly(ethylene-co-acrylic acid) available under the trade designation A-C 540, A-C 5180 and A-C 5120, poly(ethylene-co-maleic anhydride) available under the trade designation A-C 575A and A-C 573P, poly(propylene-co-maleic anhydride) available under the trade designation A-C 950P, all from Honeywell, Morristown, N.J.; poly(1-octadecene-co-maleic anhydride) available under the trade designation PA-18 from Chevron Phillips Chemical Co. LLC, The Woodlands, Tex.

The acidic polymer compound can comprise a polymer (e.g., an acidic olefin addition polymer) having an anionic group, for example a carboxylate group. In some embodiments, acidic olefin addition polymer comprising an anionic group can be referred to as an “ionomer” or a “carboxylate ionomer.” Non-limiting examples of acidic olefin addition polymers comprising an anionic group include polymers available under the trade designation ACLYN 201 and ACLYN 285, both from Honeywell, Morristown, N.J. The acidic polymer compound can comprise an oxidized olefin addition polymer.

The oxidized olefin addition polymer can be any oxidized olefin addition polymer. In some embodiments, the oxidized olefin addition polymer comprises oxidized poly(ethylene) or oxidized poly(propylene). In some embodiments, the oxidized olefin addition polymer comprises an oxidized alpha olefin polymer. Non-limiting examples of oxidized olefin addition polymers include polymers available under the trade designation A-C 645P, A-C 655, A-C 680, A-C 325, and A-C 392, all from Honeywell, Morristown, N.J., and oxidized polyolefin waxes such as those available under the trade designation LUWAX from BASF Corp., Florham Park, N.J.

The acidic polymer compound can comprise a mixture of an olefin addition polymer and a fatty carboxylic acid. In some embodiments, the acidic polymer compound can be prepared or obtained as an emulsion in water. In these embodiments, the mixture (the acidic polymer compound) can be isolated by, for example, precipitation from water using a water-soluble organic solvent, or by drying the emulsion. A non-limiting example of a useful olefin addition polymer is poly(ethylene). Non-limiting examples of fatty carboxylic acids include oleic acid, myristic acid, and stearic acid. Emulsions of mixtures of an olefin addition polymer and a fatty carboxylic acid are available under the trade designation JONCRYL 120 from BASF Corp., Florham Park, N.J.

The acidic polymer compound can comprise, in some embodiments, any one fatty carboxylic acid or a mixture of any fatty carboxylic acids. The fatty carboxylic acid can be, for example, an alkyl fatty carboxylic acid. The fatty carboxylic acid can comprise at least 6 carbon atoms, at least 8 carbon atoms, at least 10 carbon atoms, at least 12 carbon atoms, at least 14 carbon atoms, at least 16 carbon atoms, at least 18 carbon atoms, at least 20 carbon atoms, at least 22 carbon atoms, at least 24 carbon atoms, at least 26 carbon atoms, at least 28 carbon atoms, at least 30 carbon atoms, at least 32 carbon atoms, at least 34 carbon atoms, at least 36 carbon atoms, at least 38 carbon atoms, or at least 40 carbon atoms. The fatty carboxylic acid can comprise no greater than 46 carbon atoms, no greater than 40 carbon atoms, no greater than 38 carbon atoms, no greater than 36 carbon atoms, no greater than 34 carbon atoms, no greater than 32 carbon atoms, no greater than 30 carbon atoms, no greater than 28 carbon atoms, no greater than 26 carbon atoms, no greater than 24 carbon atoms, no greater than 22 carbon atoms, no greater than 20 carbon atoms, no greater than 18 carbon atoms, no greater than 16 carbon atoms, no greater than 14 carbon atoms, no greater than 12 carbon atoms, or no greater than 10 carbon atoms.

The acidic polymer compound can have a softening or melting temperature no greater than 0° C., no greater than 10° C., no greater than 20° C., no greater than 30°, no greater than 40° C., no greater than 50° C., or no greater than 60° C. The acidic polymer compound can have a softening or melting temperature of at least 20° C., at least 30° C., at least 40° C., at least 50° C., at least 60° C., at least 70° C., or at least 80° C. The term “softening temperature” refers to the temperature at which an acidic olefin addition polymer (in the form of free-flowing pellets or powder) no longer flows freely. Alternatively, the term “softening temperature” refers to the temperature at which an acidic polymer compound (in the form of, for example, a cylinder or a sheet) begins to deform (e.g., sag under its own weight) under the force of gravity.

In some embodiments, the acidic polymer compound can be crosslinked. In other embodiments, the acidic polymer compound is substantially free of crosslinks, i.e., the acidic polymer compound has no greater than 5 mole percent, no greater than 2 mole percent, no greater than 1 mole percent, no greater than 0.5 mole percent, no greater than 0.2 mole percent, no greater than 0.1 mole percent, no greater than 0.05 mole percent, no greater than 0.02 mole percent, or no greater than 0.01 mole percent crosslinks (formed by reaction of a cure site on, for example, the polymer chain or by reaction of a crosslinking agent). In still other embodiments, the acidic polymer compound is free of crosslinks.

In addition to an acidic polymer compound, the composition comprises a (meth)acrylic copolymer. The (meth)acrylic copolymer is prepared from reactants comprising at least one aliphatic, aromatic, or aralkyl meth(acrylate) monomer and at least one ethylenically unsaturated monomer having a polar group or a siloxane group.

The aliphatic, aromatic, or aralkyl meth(acrylate) monomer can comprise a compound of Formula I

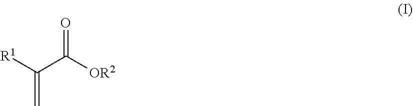

wherein R¹ comprises a hydrogen atom or an alkyl group having 1 to 4 carbon atoms, and R² comprises an alkyl, aryl, or aralkyl group having no greater than 30 carbon atoms.

In some embodiments, R¹ is a hydrogen atom (i.e., the (meth)acylate monomer comprises an acrylate monomer). In other embodiments, R¹ is an alkyl group having 1 to 4 carbon atoms. When R¹ is an alkyl group, the alkyl group can comprise a linear or branched structure. For example, R¹ can comprise a methyl group (i.e., the (meth)acrylate monomer comprises a methacrylate monomer), an ethyl group, an n-propyl group, an isopropyl group, an n-butyl group, or an isobutyl group.

In some embodiments, R² comprises a substituted or unsubstituted alkyl group. The alkyl group can comprise linear, branched, or cyclic structures. The alkyl group can comprise no greater than 26 carbon atoms, no greater than 24 carbon atoms, no greater than 22 carbon atoms, no greater than 20 carbon atoms, no greater than 18 carbon atoms, no greater than 16 carbon atoms, no greater than 14 carbon atoms, no greater than 12 carbon atoms, no greater than 10 carbon atoms, no greater than 8 carbon atoms, no greater than 6 carbon atoms, no greater than 4 carbon atoms, no greater than 2 carbon atoms, or 1 carbon atom. The alkyl group can comprise at least 28 carbon atoms, at least 26 carbon atoms, at least 24 carbon atoms, at least 22 carbon atoms, at least 20 carbon atoms, at least 18 carbon atoms, at least 16 carbon atoms, at least 14 carbon atoms, at least 12 carbon atoms, at least 10 carbon atoms, at least 8 carbon atoms, at least 6 carbon atoms, or at least 4 carbon atoms. Non-limiting examples of alkyl groups include methyl, ethyl, propyl, butyl, hexyl, octyl, decyl, dodecyl, tetradecyl, hexadecyl, octadecyl, eicosyl, docosyl, tetracosyl, hexacosyl, octacosyl, triacontyl, 2-propyl, 2-butyl, 2-hexyl, 3-octyl, 2-decyl, 4-dodecyl, cyclohexyl, and cyclohexylmethyl.

In some embodiments, R² comprises a substituted or unsubstituted aryl group. The aryl group can comprise one arene ring or more than one arene ring. Aryl groups can comprise up to 6 carbon atoms, up to 8 carbon atoms, up to 10 carbon atoms, up to 12 carbon atoms, up to 14 carbon atoms, up to 16 carbon atoms, or up to 18 carbon atoms. If more than one arene ring is present in an aryl group, the arene rings can be fused together, or they can be joined by a chemical bond. Non-limiting examples of aryl groups include substituted and unsubstituted phenyl, 1-naphthyl, 2-naphthyl, 9-anthracenyl, and biphenyl.

In some embodiments, R² comprises a substituted or unsubstituted aralkyl group. The aralkyl group can comprise one arene ring or more than one arene ring. The aralkyl group can comprise up to 6 carbon atoms, up to 8 carbon atoms, up to 10 carbon atoms, up to 12 carbon atoms, up to 14 carbon atoms, up to 16 carbon atoms, up to 18 carbon atoms, or up to 20 carbon atoms. If more than one arene ring is present in the aralkyl group, the arene rings can be fused together, or they can be joined by a chemical bond. The aralkyl group can comprise one or more alkyl groups. The alkyl group can comprise linear, branched, or cyclic structures. The alkyl groups can be bonded to an arene ring, and can comprise no greater than 20 carbon atoms, no greater than 18 carbon atoms, no greater than 16 carbon atoms, no greater than 14 carbon atoms, no greater than 12 carbon atoms, no greater than 10 carbon atoms, no greater than 8 carbon atoms, no greater than 6 carbon atoms, no greater than 4 carbon atoms, no greater than 2 carbon atoms, or 1 carbon atom. The alkyl group can comprise at least 28 carbon atoms, at least 26 carbon atoms, at least 24 carbon atoms, at least 22 carbon atoms, at least 20 carbon atoms, at least 18 carbon atoms, at least 16 carbon atoms, at least 14 carbon atoms, at least 12 carbon atoms, at least 10 carbon atoms, at least 8 carbon atoms, at least 6 carbon atoms, or at least 4 carbon atoms. Examples of alkyl groups include methyl, ethyl, 1-propyl, 2-propyl, 1-butyl, and 2-butyl groups. Non-limiting examples of aralkyl groups include benzyl, 4-methyl benzyl, 1-phenylethyl, 2-phenylethyl, 3-phenylpropyl, 2-naphthylethyl, and 9-anthracenylmethyl.

In some embodiments, the (meth)acrylate monomer comprises an alkyl (meth)acrylate monomer. In some embodiments, the alkyl(meth)acrylate monomer comprises a compound of Formula I wherein R¹ comprises a hydrogen atom or a methyl group, and R² comprises an alkyl group having 8 to 24 carbon atoms. In some embodiments, the (meth)acrylate monomer comprises isobornyl acrylate, isobornyl methacrylate, dodecyl acrylate, dodecyl methacrylate, tetradecyl acrylate, tetradecyl methacrylate, hexadecyl acrylate, hexadecyl methacrylate, octadecyl acrylate, octadecyl methacrylate, behenyl acrylate, or behenyl methacrylate.

The ethylenically unsaturated monomer having a polar group can comprise a compound of Formula II, Formula III, or Formula IV

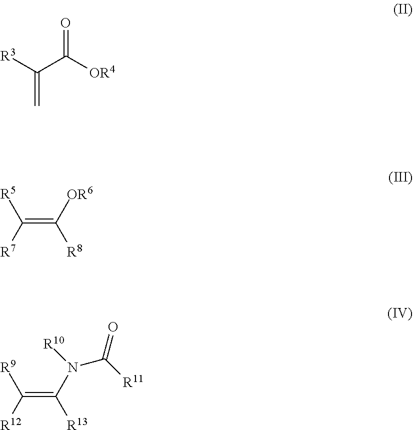

wherein R³, R⁵, R⁷, R⁸, R⁹, R¹², and R¹³ independently can comprise a hydrogen atom or an alkyl group having 1 to 4 carbon atoms. In Formula II, R⁴ can comprise a substituted or unsubstituted heteroalkyl group having 1 to 400 carbon atoms. In Formula III, R⁶ can comprise 1 to 20 carbon atoms. In Formula IV, R¹⁰ can comprise a hydrogen atom or an alkyl group having 1 to 8 carbon atoms (the alkyl group optionally substituted with a carbonyl group), and R¹¹ can comprise an alkyl group having 1 to 8 carbon atoms. Alternatively, in some embodiments R¹⁰ and R¹¹ can together form a ring structure including the nitrogen atom.

In Formulas II, III, and IV, the groups R³, R⁵, R⁷, R⁸, R⁹, R¹², and R¹³ independently comprise a hydrogen atom or an alkyl group having 1 to 4 carbon atoms. When R³, R⁵, R⁷, R⁸, R⁹, R¹², and R¹³ independently comprise an alkyl group, the alkyl group can comprise a linear or branched structure. For example, R³, R⁵, R⁷, R⁸, R⁹, R¹², and R¹³ can independently be a methyl group, an ethyl group, an n-propyl group, an isopropyl group, an n-butyl group, or an isobutyl group.

In Formula II, R⁴ can comprise a substituted or unsubstituted heteroalkyl group having 1 to 400 carbon atoms. Often, R⁴ comprises a substituted or unsubstituted heteroalkyl group having 1 to 30 carbon atoms. The heteroalkyl group (i.e., an alkyl group that comprises at least one heteroatom, e.g., oxygen, nitrogen, or sulfur) can comprise a linear, branched, or cyclic structure. The heteroalkyl group can comprise no greater than 30 carbon atoms, no greater than 28 carbon atoms, no greater than 26 carbon atoms, no greater than 24 carbon atoms, no greater than 22 carbon atoms, no greater than 20 carbon atoms, no greater than 18 carbon atoms, no greater than 16 carbon atoms, no greater than 14 carbon atoms, no greater than 12 carbon atoms, no greater than 10 carbon atoms, no greater than 8 carbon atoms, no greater than 6 carbon atoms, no greater than 4 carbon atoms, no greater than 2 carbon atoms, or 1 carbon atom. The heteroalkyl group can comprise at least 20 carbon atoms, at least 18 carbon atoms, at least 16 carbon atoms, at least 14 carbon atoms, at least 12 carbon atoms, at least 10 carbon atoms, at least 8 carbon atoms, at least 6 carbon atoms, or at least 4 carbon atoms. The heteroalkyl group can comprise no greater than 30 heteroatoms, no greater than 28 heteroatoms, no greater than 26 heteroatoms, no greater than 24 heteroatoms, no greater than 22 heteroatoms, no greater than 20 heteroatoms, no greater than 18 heteroatoms, no greater than 16 heteroatoms, no greater than 14 heteroatoms, no greater than 12 heteroatoms, no greater than 10 heteroatoms, no greater than 8 heteroatoms, no greater than 6 heteroatoms, no greater than 4 heteroatoms, no greater than 2 heteroatoms, or 1 heteroatom. The heteroalkyl group can comprise at least 22 heteroatoms, at least 20 heteroatoms, at least 18 heteroatoms, at least 16 heteroatoms, at least 14 heteroatoms, at least 12 heteroatoms, at least 10 heteroatoms, at least 8 heteroatoms, at least 6 heteroatoms, or at least 4 heteroatoms.

Non-limiting examples of heteroalkyl groups include amino groups such as 3-N,N-dimethylaminopropyl, ether groups such as methoxymethyl, and polyether groups (i.e., a group comprising more than one ether group) such as methoxyethoxyethyl and tetrahydrofurfuryl. Ether and polyether groups can comprise oxyalkylene groups, for example groups having the structure of Formula V

OCᵥH₂ᵥ_{w}, (V)

where v is an integer of 1 to 4 and w is an integer of 1 to 100. An ether group can include a group of Formula V where w is 1. Non-limiting examples of polyether groups comprising oxyalkylene groups include poly(oxymethylene), poly(oxyethylene), and poly(oxybutylene) groups. In Formula V, w can be an integer of at least 1, at least 2, at least 4, at least 6, at least 8, at least 10, at least 20, at least 30, at least 40, at least 50, at least 60, at least 80, or at least 90. In Formula V, w can be an integer of 100, no greater than 100, no greater than 80, no greater than 60, no greater than 50, no greater than 40, no greater than 20, no greater than 10, no greater than 8, no greater than 6, or no greater than 4.

In Formula III, the group R⁶ can comprise 1 to 20 carbon atoms. The group R⁶ can comprise at least 1 carbon atom, at least 2, at least 3, at least 4, at least 5, at least 6, at least 7, at least 8, at least 9, at least 10, at least 12, at least 14, at least 16, or at least 18 carbon atoms. The group R⁶ can comprise no greater than 20, no greater than 18, no greater than 16, no greater than 14, no greater than 12, no greater than 10, no greater than 9, no greater than 8, no greater than 7, no greater than 6, no greater than 5, no greater than 4, or no greater than 3 carbon atoms.

In some embodiments, R⁶ comprises an alkyl group (optionally substituted with a carbonyl group). In embodiments wherein R⁶ comprises an alkyl group, the compounds of Formula III can comprise an alkyl vinyl ether. Non-limiting examples of alkyl vinyl ethers include methyl vinyl ether and ethyl vinyl ether. In embodiments wherein the alkyl group is substituted with a carbonyl group, the compounds of Formula III can comprise a vinyl ester. Non-limiting examples of vinyl esters include vinyl acetate and vinyl propionate.

In some embodiments, R⁶ comprises a heteroalkyl group. The heteroalkyl group (i.e., an alkyl group that comprises at least one heteroatom, e.g., oxygen, nitrogen, or sulfur) can comprise a linear, branched, or cyclic structure. The heteroalkyl group can comprise no greater than 20 carbon atoms, no greater than 18 carbon atoms, no greater than 16 carbon atoms, no greater than 14 carbon atoms, no greater than 12 carbon atoms, no greater than 10 carbon atoms, no greater than 9 carbon atoms, no greater than 8 carbon atoms, no greater than 7 carbon atoms, no greater than 6 carbon atoms, no greater than 5 carbon atoms, no greater than 4 carbon atoms, no greater than 3 carbon atoms, no greater than 2 carbon atoms, or 1 carbon atom. The heteroalkyl group can comprise 16 carbon atoms, at least 14 carbon atoms, at least 12 carbon atoms, at least 10 carbon atoms, at least 9 carbon atoms, at least 8 carbon atoms, at least 7 carbon atoms, at least 6 carbon atoms, at least 5 carbon atoms, or at least 4 carbon atoms. The heteroalkyl group can comprise no greater than 20 heteroatoms, no greater than 18 heteroatoms, no greater than 16 heteroatoms, no greater than 14 heteroatoms, no greater than 12 heteroatoms, no greater than 10 heteroatoms, no greater than 9 heteroatoms, no greater than 8 heteroatoms, no greater than 7 heteroatoms, no greater than 6 heteroatoms, no greater than 5 heteroatoms, no greater than 4 heteroatoms, no greater than 3 heteroatoms, no greater than 2 heteroatoms, or 1 heteroatom. The heteroalkyl group can comprise at least 16 heteroatoms, at least 14 heteroatoms, at least 12 heteroatoms, at least 10 heteroatoms, at least 9 heteroatoms, at least 8 heteroatoms, at least 7 heteroatoms, at least 6 heteroatoms, at least 5 heteroatoms, at least 4 heteroatoms, or at least 3 heteroatoms. Non-limiting examples of heteroalkyl groups include amino groups such as 3-N,N-dimethylaminopropyl, ether groups such as methoxyethyl, and polyether groups (i.e., a group comprising more than one ether group) such as methoxyethoxyethyl and tetrahydrofurfuryl. Ether and polyether groups can comprise oxyalkylene groups, for example groups having the structure of Formula IV wherein v is an integer of 1 to 4 and w is an integer of 1 to 20.

In some embodiments, R⁶ comprises an aryl group. The aryl group can comprise at least 4 carbon atoms, at least 5 carbon atoms, at least 6 carbon atoms, at least 7 carbon atoms, at least 8 carbon atoms, at least 9 carbon atoms, at least 10 carbon atoms, at least 11 carbon atoms, or at least 12 carbon atoms. The aryl group can comprise no greater than 14 carbon atoms, no greater than 13 carbon atoms, no greater than 12 carbon atoms, no greater than 11 carbon atoms, no greater than 10 carbon atoms, no greater than 9 carbon atoms, no greater than 8 carbon atoms, no greater than 7 carbon atoms, or no greater than 6 carbon atoms. Non-limiting examples of aryl groups include phenyl, 1-naphthyl, 2-naphthyl, and 9-anthracenyl.

In some embodiments, R⁶ comprises an aralkyl group (optionally substituted with a carbonyl group). The aralkyl group can comprise at least 4 carbon atoms, at least 5 carbon atoms, at least 6 carbon atoms, at least 7 carbon atoms, at least 8 carbon atoms, at least 9 carbon atoms, at least 10 carbon atoms, at least 11 carbon atoms, or at least 12 carbon atoms. The aralkyl group can comprise no greater than 14 carbon atoms, no greater than 13 carbon atoms, no greater than 12 carbon atoms, no greater than 11 carbon atoms, no greater than 10 carbon atoms, no greater than 9 carbon atoms, no greater than 8 carbon atoms, no greater than 7 carbon atoms, or no greater than 6 carbon atoms. Non-limiting examples of aralkyl groups include benzyl, 4-methyl benzyl, 1-phenylethyl, 2-phenylethyl, 3-phenylpropyl, 2-naphthylethyl, and 9-anthracenylmethyl.

In Formula IV, R¹⁰ can comprise a hydrogen atom or an alkyl group having 1 to 8 carbon atoms (the alkyl group optionally substituted with a carbonyl group), and R¹¹ can comprise an alkyl group having 1 to 8 carbon atoms. In some embodiments, R¹⁰ comprises a hydrogen atom. Alternatively, the group R¹⁰ can comprise an alkyl group having at least 1, at least 2, at least 3, at least 4, at least 5, at least 6, at least 7, or at least 8 carbon atoms. The group R¹⁰ can comprise an alkyl group having no greater than 4, no greater than 5, no greater than 6, no greater than 7, or no greater than 10 carbon atoms. When R¹⁰ comprises an alkyl group having 1 to 8 carbon atoms, the compounds of Formula IV can be N-alkyl-N-vinyl carboxamide compounds. Non-limiting example of such compounds include N-methyl-N-vinyl acetamide and N-vinyl acetamide.

In some embodiments, R¹⁰ comprises an alkyl group substituted with a carbonyl group. The carbonyl group can be bonded (via a covalent bond) to the nitrogen atom. In embodiments wherein R¹⁰ comprises an alkyl group substituted with a carbonyl group that is bonded to the nitrogen atom, the compounds of Formula IV can be N-vinyl carboximide compounds.

In some embodiments, R¹⁰ and R¹¹ can together form a ring structure including the nitrogen atom. When R¹⁰ and R¹¹ together form a ring structure including the nitrogen atom, the ring structure comprises a N-vinyl cyclic carboxamide or (in the case where R¹⁰ comprises an alkyl group substituted with a carbonyl group) a N-vinyl cyclic carboximide. Non-limiting examples of N-vinyl cyclic carboxamides include N-vinyl pyrrolidinone and N-vinyl caprolactam. Non-limiting examples of N-vinyl cyclic carboximides include N-vinyl succinimide and N-vinyl glutarimide.

The ethylenically unsaturated monomer having a siloxane group can comprise a compound of Formula VI

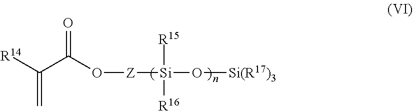

wherein R¹⁴ comprises a hydrogen atom or an alkyl group having 1 to 4 carbon atoms, Z is a divalent linking group, R¹⁵, R¹⁶, and R¹⁷ are independently alkyl groups, aryl groups, or aralkyl groups, and n is an integer of at least 1.

In Formula VI, R¹⁴ can, in some embodiments, comprise a hydrogen atom. In other embodiments, R¹⁴ comprises an alkyl group having 1 to 4 carbon atoms. When R¹⁴ is an alkyl group, the alkyl group can comprise a linear or branched structure. For example, R¹⁴ can comprise a methyl group, an ethyl group, an n-propyl group, an isopropyl group, an n-butyl group, or an isobutyl group.

The divalent linking group Z can be any divalent group. In some embodiments, the divalent linking group Z comprises at least one carbon atom bonded via a covalent bond to the silicon atom. Non-limiting examples of divalent linking groups include alkylene groups (e.g., ethylene or propylene groups), and arylene groups (e.g., a phenylene group). The alkylene groups can comprise a linear, branched, or cyclic structure. The divalent linking group Z can comprise 1 to 20 carbon atoms and can optionally include, for example, one or more ester, amide, urea, or urethane groups.

In Formula VI, R¹⁵, R¹⁶, and R¹⁷ are independently alkyl groups, aryl groups, or aralkyl groups. The alkyl group can comprise linear, branched, or cyclic structures. The alkyl group can comprise no greater than 10 carbon atoms, no greater than 8 carbon atoms, no greater than 6 carbon atoms, no greater than 4 carbon atoms, no greater than 2 carbon atoms, or 1 carbon atom. The alkyl group can comprise at least 8 carbon atoms, at least 6 carbon atoms, at least 4 carbon atoms, at least 2 carbon atoms, or at least 1 carbon atom. Non-limiting examples of alkyl groups include methyl, ethyl, propyl, butyl, hexyl, octyl, 2-propyl, 2-butyl, 2-hexyl, 3-octyl, cyclohexyl, and cyclohexylmethyl.

In Formula VI, n is an integer of at least 1, at least 2, at least 5, at least 10, at least 20, at least 30, at least 40, at least 50, at least 60, at least 70, at least 80, at least 90, or at least 100. In Formula VI, n is an integer of no greater than 5, no greater than 10, no greater than 20, no greater than 30, no greater than 40, no greater than 50, no greater than 60, no greater than 70, or no greater than 80.

In some embodiments, R¹⁵, R¹⁶, and R¹⁷ independently comprise a substituted or unsubstituted aryl group. The aryl group can comprise one arene ring or more than one arene ring. Aryl groups can comprise up to 6 carbon atoms, up to 8 carbon atoms, up to 10 carbon atoms, up to 12 carbon atoms, or up to 14 carbon atoms. If more than one arene ring is present in an aryl group, the arene rings can be fused together, or they can be joined by a chemical bond. Non-limiting examples of aryl groups include substituted and unsubstituted phenyl, 4-methylphenyl, 1-naphthyl, 2-naphthyl, 9-anthracenyl, and biphenyl.

In some embodiments, R¹⁵, R¹⁶, and R¹⁷ independently comprise a substituted or unsubstituted aralkyl group. The aralkyl group can comprise one arene ring or more than one arene ring. The aralkyl group can comprise up to 6 carbon atoms, up to 8 carbon atoms, up to 10 carbon atoms, up to 12 carbon atoms, up to 14 carbon atoms, up to 16 carbon atoms, up to 18 carbon atoms, or up to 20 carbon atoms. If more than one arene ring is present in the aralkyl group, the arene rings can be fused together, or they can be joined by a chemical bond. Non-limiting examples of aralkyl groups include benzyl, 4-methyl benzyl, 1-phenylethyl, 2-phenylethyl, 3-phenylpropyl, 2-naphthylethyl, and 9-anthracenylmethyl.

Representative examples of compounds of Formula VI include, for example, methacryloxypropyl-terminated poly(dimethylsiloxane).

The (meth)acrylic copolymer can have a weight average molecular weight of at least 5,000, at least 10,000, at least 25,000, at least 50,000, at least 75,000, at least 100,000, at least 150,000, at least 200,000, at least 250,000, at least 300,000, at least 350,000, at least 400,000, at least 450,000, at least 500,000, at least 550,000, at least 600,000, at least 650,000, at least 700,000, at least 750,000, at least 800,000, at least 850,000, at least 900,000, at least 950,000, or at least 1,000,000. The (meth)acrylic copolymer can have a weight average molecular weight of no greater than 20,000, no greater than 25,000, no greater than 50,000, no greater than 75,000, no greater than 100,000, no greater than 150,000, no greater than 200,000, no greater than 250,000, no greater than 300,000, no greater than 350,000, no greater than 400,000, no greater than 450,000, no greater than 500,000, no greater than 550,000, no greater than 600,000, no greater than 650,000, or no greater than 700,000.

The (meth)acrylic copolymer can have a glass transition temperature (T_{g}) of at least −100° C., at least −80° C., at least −70° C., at least −60° C., at least −50° C., at least −40° C., at least −30° C., at least −20° C., at least −10° C., at least 0° C., at least 10° C., at least 20° C., at least 30° C., or at least 40° C. The (meth)acrylic copolymer can have a glass transition temperature (T_{g}) of no greater than −80° C., no greater than −70° C., no greater than −60° C., no greater than −50° C., no greater than −40° C., no greater than −30° C., no greater than −20° C., no greater than −10° C., no greater than 0° C., no greater than 10° C., no greater than 20° C., no greater than 30° C., no greater than 40° C., no greater than 50° C., or no greater than 60° C.

In some embodiments, the (meth)acrylic copolymer is a pressure sensitive adhesive. In this context, the term “pressure sensitive adhesive” refers to a (meth)acrylic copolymer (or to a composition comprising a (meth)acrylic copolymer) with properties including aggressive and persistent tack, adherence with no more than finger pressure, sufficient ability to hold onto an adherent, sufficient cohesive strength, and no activation by an energy source. Pressure sensitive adhesives are typically tacky at temperatures at or above room temperature (i.e., at or above about 20° C. to about 30° C. or greater).

In some embodiments, the (meth)acrylic copolymer comprises a linear (meth)acrylic copolymer, i.e., a (meth)acrylic copolymer comprising a linear polymer chain structure. In some embodiments, the (meth)acrylic copolymer comprises a branched structure. In some embodiments, the (meth)acrylic copolymer is substantially free of branching (i.e., the (meth)acrylic copolymer comprises polymer chains having no greater than one branching point along the main polymer chain). Typically, the (meth)acrylic copolymer is free of core/shell structure (i.e., the (meth)acrylic copolymer does not comprise a core/shell polymer).

In some embodiments, (meth)acrylic copolymer can be crosslinked. In other embodiments, the (meth)acrylic copolymer is substantially free of crosslinks, i.e., the (meth)acrylic copolymer has no greater than 5 mole percent, no greater than 2 mole percent, no greater than 1 mole percent, no greater than 0.5 mole percent, no greater than 0.2 mole percent, no greater than 0.1 mole percent, no greater than 0.05 mole percent, no greater than 0.02 mole percent, or no greater than 0.01 mole percent crosslinks (formed by reaction of a cure site on the polymer chain or by reaction of a crosslinking agent). In still other embodiments, the (meth)acrylic copolymer is free of crosslinks.

The composition can comprise any weight percentage of the acidic polymer compound, based on the combined weights of the acidic polymer compound and the (meth)acrylic copolymer. The composition can comprise at least 5 weight percent, at least 10 weight percent, at least 20 weight percent, at least 30 weight percent, at least 40 weight percent, at least 50 weight percent, at least 60 weight percent, or at least 70 weight percent of the acidic polymer compound, based on the combined weights of the acidic polymer compound and the (meth)acrylic copolymer. The composition can comprise no greater than 95 weight percent, no greater than 90 weight percent, no greater than 80 weight percent, no greater than 70 weight percent, no greater than 60 weight percent, no greater than 50 weight percent, no greater than 40 weight percent, no greater than 30 weight percent, no greater than 20 weight percent, or no greater than 10 weight percent of the acidic polymer compound, based on the combined weights of the acidic polymer compound and the (meth)acrylic copolymer. The composition can comprise one acidic polymer compound or more than one acidic polymer compound.

The composition can comprise any weight percentage of the (meth)acrylic copolymer, based on the combined weights of the acidic polymer compound and the (meth)acrylic copolymer. The composition can comprise at least 5 weight percent, at least 10 weight percent, at least 20 weight percent, at least 30 weight percent, at least 40 weight percent, at least 50 weight percent, at least 60 weight percent, or at least 70 weight percent of the (meth)acrylic copolymer, based on the combined weights of the acidic polymer compound and the (meth)acrylic copolymer. The composition can comprise no greater than 95 weight percent, no greater than 90 weight percent, no greater than 80 weight percent, no greater than 70 weight percent, no greater than 60 weight percent, no greater than 50 weight percent, no greater than 40 weight percent, no greater than 30 weight percent, no greater than 20 weight percent, or no greater than 10 weight percent of the (meth)acrylic copolymer, based on the combined weights of the acidic polymer compound and the (meth)acrylic copolymer. The composition can comprise one (meth)acrylic copolymer or more than one (meth)acrylic copolymer.

The acidic polymer compound and the (meth)acrylic copolymer can be compatible. In this context, the term “compatible” refers to a tendency of a mixture of the acidic polymer compound and the (meth)acrylic copolymer to be macroscopically homogeneous. That is, the mixture appears to be homogeneous (i.e., a single phase) when observed using the unaided eye. In some embodiments, the mixture appears to be homogeneous when observed using an optical microscope. In other embodiments, the mixture appears to be homogeneous when observed using an electron microscope.

The acidic polymer compound can dissolve in the (meth)acrylic copolymer to form a solution of the acidic polymer compound in the (meth)acrylic copolymer. At least 10 weight percent, at least 20 weight percent, at least 30 weight percent, at least 40 weight percent, at least 50 weight percent, at least 60 weight percent, or at least 70 weight percent of the acidic polymer compound can dissolve in the (meth)acrylic copolymer in the composition. No greater than 95 weight percent, no greater than 90 weight percent, no greater than 80 weight percent, no greater than 70 weight percent, no greater than 60 weight percent, no greater than 50 weight percent, no greater than 40 weight percent, no greater than 30 weight percent, or no greater than 20 weight percent of the acidic polymer compound can dissolve in the (meth)acrylic copolymer in the composition.

The (meth)acrylic copolymer can dissolve in the acidic polymer compound to form a solution of the (meth)acrylic copolymer in the acidic polymer compound. At least 10 weight percent, at least 20 weight percent, at least 30 weight percent, at least 40 weight percent, at least 50 weight percent, at least 60 weight percent, at or least 70 weight percent of the (meth)acrylic copolymer can dissolve in the acidic polymer compound in the composition. No greater than 95 weight percent, no greater than 90 weight percent, no greater than 80 weight percent, no greater than 70 weight percent, no greater than 60 weight percent, no greater than 50 weight percent, no greater than 40 weight percent, no greater than 30 weight percent, or no greater than 20 weight percent of the (meth)acrylic copolymer can dissolve in the acidic polymer compound in the composition. In some embodiments, the acidic polymer compound and the (meth)acrylic copolymer are miscible.

The acidic polymer compound and the (meth)acrylic copolymer can react with each other to form, for example, ionic bonds or covalent bonds. Ionic or covalent bonds between the acidic polymer compound and the (meth)acrylic copolymer can be crosslinks and can form a crosslinked network wherein the acidic polymer compound is bonded to the (meth)acrylic copolymer via more than one ionic or covalent bond. Typically, the acidic polymer compound and the (meth)acrylic copolymer do not react with each other to form, for example, ionic bonds or covalent bonds. In some embodiments, the acidic polymer compound comprises organic functional groups that are capable of reacting with organic functional groups on the (meth)acrylic copolymer to form ionic bonds or covalent bonds, but these functional groups typically do not react with each other under conditions of, for example, temperatures reached during processing or use of the compositions. In some embodiments, the composition is substantially free of ionic or covalent bonds between the acidic polymer compound and the (meth)acrylic copolymer. The term “substantially free of ionic or covalent bonds” refers to a composition in which at least one of the acidic polymer compound or the polymer can be dissolved in a solvent to form a solution of at least one of the acidic polymer compound or the (meth)acrylic copolymer in the solvent. In some embodiments, the composition is free of ionic or covalent bonds between the acidic olefin addition polymer and the (meth)acrylic copolymer.

The acidic polymer compound and the (meth)acrylic copolymer can react with each other to form hydrogen bonds. The acidic polymer compound and the (meth)acrylic copolymer can independently comprise a hydrogen bond donor (e.g., a hydrogen atom that is covalently bonded to an oxygen atom or a nitrogen atom) or a hydrogen bond acceptor (e.g., an oxygen atom or a nitrogen atom). In some embodiments, the composition is substantially free of hydrogen bonds between the acidic polymer compound and the (meth)acrylic copolymer. The term “substantially free of hydrogen bonds” refers to a composition in which at least one of the acidic polymer compound or the (meth)acrylic copolymer can be dissolved in a solvent comprising a hydrogen bond donor or a hydrogen bond acceptor to form a solution of at least one of the acidic polymer compound or the (meth)acrylic copolymer in the solvent. In some embodiments, the composition is free of hydrogen bonds between the acidic polymer compound and the (meth)acrylic copolymer.

The composition can comprise a crosslinking agent. A crosslinking agent can link together (i.e., can form hydrogen bonds, ionic bonds, or covalent bonds with) at least two polymer chains. For example, a crosslinking agent can link together at least two acidic olefin addition polymer chains or at least two meth(acrylic) copolymer chains. In some embodiments, a crosslinking agent can link together an acidic olefin addition polymer chain and a (meth)acrylic copolymer chain. In some embodiments, the composition comprises less than 10 weight percent crosslinking agent, based on the combined weights of the acidic polymer compound and the (meth)acrylic copolymer. In some embodiments, the composition is substantially free of crosslinking agent, i.e., it comprises less than 8 weight percent, less than 6 weight percent, less than 4 weight percent, less than 2 weight percent, less than 1 weight percent, less than 0.5 weight percent, less than 0.2 weight percent, less than 0.1 weight percent, or less than 0.05 weight percent crosslinking agent, based on the combined weights of the of the acidic polymer compound and the (meth)acrylic copolymer. In some embodiments, the composition is free of crosslinking agent.

The composition can comprise additional components such as fillers, dyes, pigments, flavoring agents, or medicaments such as anticaries agents (e.g., fluoride sources) or antibiotics.

The composition can comprise a polyterpene such as gutta percha. In some embodiments, the composition is substantially free of gutta percha. In this context.

“substantially free of gutta percha” refers to a composition comprising less than 15 weight percent, less than 10 weight percent, less than 5 weight percent, less than 2 weight percent, less than 1 weight percent, or less than 0.5 weight percent gutta percha. In some embodiments, the composition is free of gutta percha.

The composition can comprise at least one filler. A filler can be an inorganic filler comprising an oxide of silicon (silica) or an oxide of zirconium (zirconia), and can further comprise oxides of other chemical elements such yttrium. Suitable silicas include fumed silica and nanoparticulate silica. Suitable zirconias include nanoparticulate zirconias. In some embodiments, the fillers are surface-modified inorganic fillers (i.e., inorganic fillers modified with organic groups). Suitable inorganic fillers are described in, for example, U.S. Patent Application Publication No. 2005/0256223 (Kolb, et al.) and U.S. Pat. Nos. 6,387,981 (Zhang et al.), 6,572,693 (Wu et al.), 7,090,721 (Craig et al.), and 7,156,911 (Kangas et al.).

The composition can comprise at least 1 weight percent, at least 2 weight percent, at least 5 weight percent, at least 10 weight percent, at least 15 weight percent, at least 20 weight percent, at least 25 weight percent, at least 30 weight percent, at least 35 weight percent, at least 40 weight percent, at least 45 weight percent, at least 50 weight percent, at least 55 weight percent, at least 60 weight percent, at least 65 weight percent, or at least 70 weight percent inorganic filler, based on the total weight of the composition. The composition can comprise no greater than 10 weight percent, no greater than 15 weight percent, no greater than 20 weight percent, no greater than 25 weight percent, no greater than 30 weight percent, no greater than 35 weight percent, no greater than 40 weight percent, no greater than 45 weight percent, no greater than 50 weight percent, no greater than 55 weight percent, no greater than 60 weight percent, no greater than 65 weight percent, no greater than 70 weight percent, no greater than 75 weight percent, no greater than 80 weight percent, or no greater than 85 weight percent inorganic filler, based on the total weight of the composition.

In some embodiments, the fillers comprise radiopaque inorganic fillers such as various barium compounds (e.g., barium sulfate, barium ziconate, barium strontium titanium oxide, or barium tungstate) or oxides of zirconium (including yttrium-containing oxides of zirconium). The fillers can comprise at least 1 weight percent, at least 2 weight percent, at least 5 weight percent, at least 10 weight percent, at least 15 weight percent, at least 20 weight percent, at least 25 weight percent, at least 30 weight percent, at least 35 weight percent, at least 40 weight percent, at least 45 weight percent, at least 50 weight percent, at least 55 weight percent, at least 60 weight percent, at least 65 weight percent, or at least 70 weight percent radiopaque filler, based on the total weight of the filler in the composition. The fillers can comprise no greater than 5 weight percent, no greater than 10 weight percent, no greater than 15 weight percent, no greater than 20 weight percent, no greater than 25 weight percent, no greater than 30 weight percent, no greater than 35 weight percent, no greater than 40 weight percent, no greater than 45 weight percent, no greater than 50 weight percent, no greater than 55 weight percent, no greater than 60 weight percent, no greater than 65 weight percent, no greater than 70 weight percent, no greater than 75 weight percent, no greater than 80 weight percent, no greater than 85 weight percent, no greater than 90 weight percent, no greater than 95 weight percent, or no greater than 98 weight percent radiopaque filler, based on the total weight of the filler in the composition.

The composition can be flexible. As used herein, the term “flexible” means that the composition can be deformed (e.g., bent, compressed, or stretched) without breaking at temperatures greater than room temperature. The composition can be sufficiently flexible or deformable such that it is capable of being inserted into a dental cavity, e.g., into a root canal. In some embodiments, a sample of the composition can be stretched to at least 100% of its length without breaking. In some embodiments, the composition is flexible at the normal temperature of the human body (i.e., approximately 37° C.). The composition can be flexible at temperatures of up to 40° C., up to 50° C., up to 60° C., up to 70° C., or up to 80° C.

In some embodiments, the composition can have a melting point of no greater than 80° C. In this context, the term “melting point” refers to a temperature at which the composition becomes liquid or liquid-like (i.e., it can flow, e.g., into a root canal, under the force of gravity). The composition can have a melting point of no greater than 60° C., no greater than 50° C., no greater than 40° C., no greater than 37° C., or no greater than 35° C. The composition can have a melting point of at least 35° C., at least 37° C., at least 40° C., at least 50° C., at least 60° C., at least 70° C., or at least 80° C.

The composition can be radiopaque, i.e., it can absorb as much X-ray radiation as an equivalent thickness of aluminum. In some embodiments, the composition is more radiopaque than tooth enamel. In some embodiments, the composition is more radiopaque than dentin. A cross-section of the composition can have radiopacity less than, equal to, or greater than the radiopacity of an equivalent cross-section of aluminum. The radiopacity of the composition can be measured as described in, for example, ISO 4049 §7.14 (2000).

The composition can be prepared by combining an acidic polymer compound and a (meth)acrylic copolymer. The (meth)acrylic copolymer is prepared from reactants comprising at least one aliphatic, aromatic, or aralkyl meth(acrylate) monomer and at least one ethylenically unsaturated monomer having a polar group or a siloxane group. The (meth)acrylic copolymer has a glass transition temperature no greater than 20° C. In some embodiments, the components or the mixture can be heated. The components or the mixture can be heated to at least any temperature sufficient to provide a mixture with sufficiently low viscosity to allow mixing by any conventional mixing method (e.g., hand mixing or mechanical mixing). The mixture can be formed into a useful shape, for example by extruding or by molding, before or after it is allowed to cool.

A method is provided for restoring a dental cavity, comprising providing a composition comprising an acidic polymer compound and a (meth)acrylic copolymer, and inserting the composition into the dental cavity. The (meth)acrylic copolymer is prepared from reactants comprising at least one aliphatic, aromatic, or aralkyl meth(acrylate) monomer and at least one ethylenically unsaturated monomer having a polar group or a siloxane group. The (meth)acrylic copolymer has a glass transition temperature no greater than 20° C. The dental cavity can be a root canal. The composition can further comprise a filler. The filler can be a radiopaque filler.

The method can comprise inserting the composition into the dental cavity. The dental cavity, e.g., a root canal, can be shaped with hand tools or rotary tools such as files before the composition is inserted into the cavity. In some embodiments, the dental cavity is not shaped before the composition is inserted. The composition can adapt to the contours of the dental cavity. In some embodiments, the composition fills the dental cavity. The method can further comprise compacting the composition in the dental cavity. When the dental cavity is a root canal, the composition can be compacted toward the apex of the canal and can provide an apical seal. In some embodiments, the composition can be injected, for example through a hollow needle or a canula, into a root canal.

In some embodiments, the method comprises heating the composition, e.g., to soften it before inserting it into a dental cavity. The composition can be heated to a temperature greater than room temperature (i.e. greater than about 20° C.). The composition can be heated to at least 20° C., at least 30° C., at least 40° C., at least 50° C., or at least 60° C. to soften it before inserting it into a dental cavity. The composition can be heated to a temperature of no greater than 80° C., no greater than 70° C., no greater than 60° C., no greater than 50° C., no greater than 40° C., or no greater than 30° C. to soften it before inserting it into a dental cavity.

In some embodiments, the composition is heated to a temperature equal to or greater than its melting point or its softening point before it is inserted into a dental cavity. In these embodiments, the dental cavity can be filled by allowing the composition to flow into the dental cavity.

The composition can flow or can be compacted to conform to the contours of the dental cavity, e.g., the root canal. Surprisingly, the composition can conform to the contours of the dental cavity and provide a seal along the contours of the dental cavity. In some embodiments, a dental cavity can be filled with the composition without the use of an additional sealing agent such as zinc oxide eugenol sealing agents.

An article is provided, comprising an acidic polymer compound and a (meth)acrylic copolymer. The (meth)acrylic copolymer is prepared from reactants comprising at least one aliphatic, aromatic, or aralkyl meth(acrylate) monomer and at least one ethylenically unsaturated monomer having a polar group or a siloxane group. The (meth)acrylic copolymer has a glass transition temperature no greater than 20° C. The article can have any shape or aspect ratio, including a shape or an aspect ratio of a root canal. In this context, the term “aspect ratio” means the ratio of the length of the article to the width of the article. In the case of an article having a tapered or conical shape, the width is the widest width of the article. The article can have an aspect ratio of at least 1:1, at least 2:1, at least 3:1, at least 4:1, at least 5:1, at least 10:1, at least 20:1, at least 30:1, at least 40:1, at least 50:1, at least 60:1, at least 70:1, or at least 80:1. The article can have an aspect ratio no greater than 80:1, no greater than 70:1, no greater than 60:1, no greater than 50:1, no greater than 40:1, no greater than 30:1, no greater than 20:1, no greater than 10:1, no greater than 5:1, no greater than 4:1, no greater than 3:1, or no greater than 2:1. In some embodiments, the article has a shape of a cylinder or cone. At least one cylinder or cone can be inserted into a dental cavity, e.g., a root canal. At least one cylinder or cone can fill the dental cavity. The cylinder or cone can have a unitary construction. Alternatively, the cylinder or cone can comprise a flexible or rigid core or carrier that is at least partially covered with a composition comprising an acidic polymer compound and a (meth)acrylic copolymer, the (meth)acrylic copolymer prepared from reactants comprising at least one aliphatic, aromatic, or aralkyl meth(acrylate) monomer and at least one ethylenically unsaturated monomer having a polar group or a siloxane group.

The article (in the shape of a cylinder or cone) can be inserted into a dental cavity (e.g., a root canal) in one piece. In some embodiments, the article can be inserted into a dental cavity in more than one piece. The article can be heated, for example by using a heated wire, after it is inserted into a dental cavity.

The article can be removed from a dental cavity. The article can comprise a composition having sufficient mechanical strength so that the article can be removed from a dental cavity in one piece (i.e., without breaking). In some embodiments, an article can be removed from a dental cavity in more than one piece. The article can be heated to a temperature at or above the melting point of the composition, and can then be removed from a dental cavity using, for example, suction via a canula. In some embodiments, the article is broken into pieces or ground into particles or a powder (e.g., using a rotary or hand tool) before it is removed from a dental cavity.

### EXAMPLES

Unless otherwise noted, reagents and solvents were or can be obtained from Sigma-Aldrich Co., St. Louis, Mo.

“IBMA” refers to isobornyl methacylate.

“ODA” refers to octadecyl acrylate

“MOEA” refers to 2-methoxyethyl acrylate, obtained from Polysciences, Inc., Warrington, Pa.

“NVP” refers to N-vinyl-2-pyrrolidinone.

“VAZO” refers to 2,2′-azobis(2,4-dimethylvaleronitrile), available under the trade designation VAZO 52 from E.I. du Pont de Nemours and Company, Wilmington, Del.

“GP-496” refers to an epoxy-functional silicone copolymer available from Genesee Polymers Corp., Burton, Mich.

“POLY(ODA)” refers to poly(octadecyl acrylate obtained from Scientific Polymer Products Inc., Ontario, N.Y.

“J120” refers to a poly(ethylene) wax and fatty carboxylic acid mixture obtained as an aqueous emulsion under the trade designation JONCRYL 120 from BASF Corp., Florham Park, N.J. The wax was precipitated by adding the emulsion to ethanol, filtering the precipitate, washing the precipitate with water, and drying the precipitate in air at room temperature. The dry solid was then ground into a fine powder.

“A-C285” refers to a low molecular weight ionomer obtained under the trade designation “ACLYN 285” from Honeywell International, Inc., Morristown, N.J.

“A-C5180” refers to poly(ethylene-co-acrylic acid), obtained under the trade designation A-C 5180 from Honeywell International, Inc., Morristown, N.J.

“A-C5120” refers to poly(ethylene-co-acrylic acid), obtained under the trade designation A-C 5180 from Honeywell International, Inc., Morristown, N.J.

“A-C645P” refers to an oxidized copolymer obtained under the trade designation A-C 645P from Honeywell International, Inc., Morristown, N.J.

“A-C575A” refers to poly(ethylene-co-maleic anhydride) available under the trade designation A-C 575A from Honeywell International, Inc., Morristown, N.J.

“EMAA-12” refers to poly(ethylene-co-methacrylic acid), having 12% methacrylic acid, available from Scientific Polymer Products Inc., Ontario, N.Y.

“PA-18” refers to poly(1-octadecene-co-maleic anhydride), available under the trade designation PA-18 from Chevron Phillips Chemical Co. LLC, The Woodlands, Tex.

“PVS” refers to poly(vinyl stearate), available from Scientific Polymer Products Inc., Ontario, N.Y.

“SYNCROWAX” refers to a mixture of fatty carboxylic acid obtained under the trade designation SYNCROWAX AWl-C from Croda Inc., Edison, N.J.

“FILLER A” refers to nanoparticulate zirconia obtained from Sigma-Aldrich Co., St. Louis, Mo.

“FILLER B” refers to barium ziconate obtained from Sigma-Aldrich Co., St. Louis, Mo.

“FILLER C” refers to nanoparticulate barium strontium titanium oxide obtained from Sigma-Aldrich Co., St. Louis, Mo.

“FILLER D” refers to zirconium (IV) oxide-yttria stabilized nanopowder, obtained from Sigma-Aldrich Co., St. Louis, Mo.\

“FILLER E” refers to stearic acid-treated zirconia prepared by treating a zirconia sol with stearic acid at room temperature overnight. The solvent was removed and the dry solid filler was ground using a mortar and pestle.

### Preparative Example 1

### Preparation of a (Meth)Acrylic Copolymer

Ten grams of a mixture of IBMA (3 parts by weight), ODA (3 parts by weight), MOEA (1 part by weight), NVP (1 part by weight), and VAZO (0.15 g) was placed in a screw cap vial. The vial was placed in a water bath at 50° C. After 8 hours, the vial was removed from the water bath and was allowed to cool to room temperature to afford the product.

### Example 1

A mixture of A-C5120 (2 g), the product of Preparative Example 1 (1.5 g), Filler B (1 g), and Filler E (0.5 g) in a test tube was heated in a block on a thermostatically controlled hot plate (temperature set to 100° C. to 150° C.) for approximately 30 minutes. The softened mixture was then immediately poured into the barrel of a glass syringe. The syringe plunger was then inserted into the barrel and the softened mixture was expelled through the tip of the syringe into cold (approximately 0° C.) 95% ethanol in an aluminum dish. The expelled mixture was then cut into pieces (approximately 5 cm to approximately 10 cm in length) and the pieces were allowed to dry at room temperature.

### Example 2

A mixture of A-C5120 (2 g), the product of Preparative Example 1 (1.5 g), Filler A (0.5 g), and Filler E (1 g) in a test tube was heated in a block on a thermostatically controlled hot plate (temperature set to 100° C. to 150° C.) for approximately 30 minutes. The softened mixture was then immediately poured into the barrel of a glass syringe. The syringe plunger was then inserted into the barrel and the softened mixture was expelled through the tip of the syringe into cold (approximately 0° C.) 95% ethanol in an aluminum dish. The expelled mixture was then cut into pieces (approximately 5 cm to approximately 10 cm in length) and the pieces were allowed to dry at room temperature.

### Examples 3-6

To prepare the compositions of Examples 3-6, J120, the product of Preparative example 1, a filler, and an additional component were combined in a test tube and the test tube was placed in a block on a thermostatically controlled hot plate (temperature set to 100° C. to 150° C.) for approximately 30 minutes. The softened mixture was then immediately poured into the barrel of a glass syringe. The syringe plunger was then inserted into the barrel and the softened mixture was expelled through the tip of the syringe into cold (approximately 0° C.) 95% ethanol in an aluminum dish. The expelled mixture was then cut into pieces (approximately 5 cm to approximately 10 cm in length) and the pieces were allowed to dry at room temperature. The components and amounts of each of the compositions of Examples 3-6 are given in Table 1. In Table 1, “PE1” refers to the product of Preparative Example 1.

TABLE 1
Compositions of Examples 3-6.

| EXAMPLE | J120 | | PE1 | | ADDITIONAL COMPONENT | FILLER |
|---|---|---|---|---|---|---|
| 3 | 2 | g | 0.5 | g | GP-496 (1 g) | FILLER C (1.5 g) |
| 4 | 1.5 | g | 1 | g | SYNCROWAX (0.5 g) | FILLER C (2 g) |
| 5 | 1.5 | g | 1 | g | PVS (0.5 g) | FILLER C (2 g) |
| 6 | 1.5 | g | 1 | g | POLY(ODA) (0.5 g) | FILLER D (2 g) |

### Examples 7-12

The compositions of Examples 7-12 were prepared using the procedure essentially as described in Examples 1-6. In Examples 7-12, each additional component, if present, comprises an acidic olefin addition polymer. The components and amounts of each of the compositions of Examples 7-12 are given in Table 2. In Table 2, “PE1” refers to the product of Preparative Example 1, and “---” means that an additional component was not present in the composition.

TABLE 2
Compositions of Examples 7-12.

| EXAMPLE | J120 | PE1 | ADDITIONAL COMPONENT | FILLER |
|---|---|---|---|---|
| 7 | 1.5 g | 1 g | EMAA-12 (0.5 g) | FILLER C (2 g) |
| 8 | 1.5 g | 1 g | PA-18 (0.5 g) | FILLER C (2 g) |
| 9 | 1.5 g | 1 g | A-C575A (0.5 g) | FILLER C (2 g) |
| 10 | 1.5 g | 1 g | A-C645P (0.5 g) | FILLER C (2 g) |
| 11 | 2.5 g | 1 g | — | FILLER C (1.5 g) |
| 12 | 2.5 g | 1 g | — | FILLER B (1.5 g) |

The complete disclosures of the patents, patent documents, and publications cited herein are incorporated by reference in their entirety as if each were individually incorporated. Various modifications and alterations to this invention will become apparent to those skilled in the art without departing from the scope and spirit of this invention. It should be understood that this invention is not intended to be unduly limited by the illustrative embodiments and examples set forth herein and that such examples and embodiments are presented by way of example only with the scope of the invention intended to be limited only by the claims set forth herein as follows.

1. A composition comprising:
a) an acidic polymer compound; and
b) a (meth)acrylic copolymer prepared from reactants comprising:
i) at least one aliphatic, aromatic, or aralkyl meth(acrylate) monomer; and
ii) at least one ethylenically unsaturated monomer having a polar group or a siloxane group,
wherein the (meth)acrylic copolymer has a glass transition temperature no greater than 60° C.

2. The composition of claim 1 wherein the (meth)acrylic copolymer has a glass transition temperature no greater than 20° C.

3. The composition of claim 1 wherein the (meth)acrylic copolymer is prepared from at least one aliphatic meth(acrylate) monomer, and the aliphatic meth(acrylate) monomer comprises an alkyl group having 4 to 18 carbon atoms.

4. The composition of claim 1 wherein the acidic polymer compound comprises a polymer prepared from reactants comprising ethylene or an alpha-olefin.

5. The composition of claim 1 wherein the acidic polymer compound comprises a polymer prepared from reactants comprising at least one acidic monomer or at least one acid-precursor monomer.

6. The composition of claim 1 wherein the acidic polymer compound comprises an oxidized olefin addition polymer.

7. A composition comprising:
a) an acidic polymer compound comprising a polymer prepared from reactants comprising ethylene or an alpha-olefin; and
b) a (meth)acrylic copolymer prepared from reactants comprising:
i) at least one C8 to C18 alkyl meth(acrylate) monomer; and
ii) at least one ethylenically unsaturated monomer having a polar group or a siloxane group,
wherein the (meth)acrylic copolymer has a glass transition temperature no greater than 60° C.

8. The composition of claim 1 wherein the alkyl (meth)acrylate monomer comprises at least one of isobornyl acrylate, isobornyl methacrylate, octadecyl acrylate, or lauryl methacrylate.

9. The composition of claim 1 further comprising at least one C4 to C18 (meth)acrylic homopolymer.

10. The composition of claim 1 further comprising at least one polymer prepared from reactants comprising a vinyl ester of a C8 to C20 alkanoic acid.

11. The composition of claim 1 wherein the composition is substantially free of a crosslinking agent.

12. The composition of claim 1 wherein the composition is substantially free of crosslinks.

13. The composition of claim 1 further comprising a filler.

14. The composition of claim 13 wherein the filler comprises a filler having a primary particle size no greater than 100 nanometers.

15. The composition of claim 13 wherein the filler is radiopaque.

16. The composition of claim 1 wherein the composition is radiopaque.

17. A method of restoring a dental cavity comprising:
a) providing a composition comprising:
a) an acidic polymer compound; and
b) a (meth)acrylic copolymer prepared from reactants comprising:
i) at least one aliphatic, aromatic, or aralkyl meth(acrylate) monomer; and
ii) at least one ethylenically unsaturated monomer having a polar group or a siloxane group,
wherein the (meth)acrylic copolymer has a glass transition temperature no greater than 60° C.; and
b) inserting the composition into the dental cavity.

18. The method of claim 17 wherein the composition further comprises a radiopaque filler having a primary particle size no greater than 100 nanometers.

19. The method of claim 17 further comprising heating the composition.

20. An article for filling a root canal comprising a composition comprising:
a) an acidic polymer compound; and
b) a (meth)acrylic copolymer prepared from reactants comprising:
i) at least one aliphatic, aromatic, or aralkyl meth(acrylate) monomer; and
ii) at least one ethylenically unsaturated monomer having a polar group or a siloxane group,
wherein the (meth)acrylic copolymer has a glass transition temperature no greater than 60° C.,
wherein the article has an aspect ratio of at least 2 to 1.
